(19) United States
(12) Patent Application Publication
(10) Pub. No.: US 2013/0191934 A1
(43) Pub. Date: Jul. 25, 2013

(54) TREATMENT OF SYMPTOMS ASSOCIATED WITH MENOPAUSE

(71) Applicants: Anantha Shekhar, Indianapolis, IN (US); Philip L. Johnson, Indianapolis, IN (US)

(72) Inventors: Anantha Shekhar, Indianapolis, IN (US); Philip L. Johnson, Indianapolis, IN (US)

(73) Assignee: INDIANA UNIVERSITY RESEARCH AND TECHNOLOGY CORPORA, INDIANAPOLIS, IN (US)

(21) Appl. No.: 13/876,053

(22) PCT Filed: Sep. 29, 2011
(86) PCT No.: PCT/US11/54020

§ 371 Date: Mar. 26, 2013

Related U.S. Application Data
(60) Provisional application No. 61/388,960, filed on Oct. 1, 2010.
(60) Provisional application No. 61/477,895, filed on Apr. 21, 2011.

(51) Int. Cl. A61K 31/551; A61K 31/47; A61K 31/4375

(52) U.S. Cl. CPC A61K 31/551; A61K 31/4375; A61K 31/47
USPC 800/9; 514/300; 514/218; 514/307

(57) ABSTRACT

A method for treating symptoms associated with a dramatic reduction in reproductive hormone levels is provided, particularly in menopausal women and breast cancer survivors undergoing aromatase inhibitor therapy. The method comprises administered to a subject an inhibitor of orexin activity in an amount sufficient to reduce or decrease onset, progression, severity, frequency, duration or probability of one or more such symptoms. A method of detecting compounds having activity for relieving menopausal symptoms is also provided.

### CROSS REFERENCE TO RELATED APPLICATIONS

This application claims priority under 35 USC §119(e) to U.S. provisional application Ser. No. 61/388,960 filed on Oct. 1, 2010, and U.S. provisional application Ser. No. 61/477,895 filed on Apr. 21, 2011, the entire disclosures of each of which are incorporated herein by reference

### TECHNICAL FIELD

The present disclosure pertains generally to the field of neuroscience. More specifically, the present disclosure pertains to the treatment of symptoms associated with dramatic reduction in reproductive hormones (for example, hot flashes/hormonal disturbances and sleep disturbance) that are common in women during menopause and following breast cancer treatments that inhibit reproductive hormone activity, but are also present postpartum, and pre-menstrual.

### BACKGROUND

Menopause represents the transition of a woman from a reproductive to non-reproductive state as a result of a major reduction in female hormonal production by the ovaries. The transition is a natural process or may be the result of surgical intervention (removal of the ovaries) or the result of therapeutic regiment (e.g., administration of aromatase inhibitors to women with estrogen receptor positive breast cancer).

Menopausal symptoms affect about 70% of women approaching menopause. Typical menopause symptoms last for the whole menopause transition, but some women may experience them for the rest of their lives. The most common symptoms are: hot flashes, night sweats, irregular periods, loss of libido, sleep disturbance and vaginal dryness. However, “hot flashes” are the most common symptom and 75% of postmenopausal women surveyed reported repeated “hot flash” episodes over and average of 3.8 years following onset of menopause. Hot flashes (which are immediately followed by a decrease in core body temp) are a result of blood flow to skin and sudomotor sweat responses that raise skin temp to reduce core body temperature. Although no single well-defined menopausal symptom cluster has emerged from published literature, data suggest that various combinations of these symptoms are common.

Although hormone therapy alleviates menopausal symptoms, there have been longstanding contraindications against its use in breast cancer survivors. In addition, many other currently available pharmacological and alternative treatments are unacceptable, poorly tolerated, or show limited efficacy. Thus additional non-hormonal treatments are needed. Unfortunately, it is difficult to identify potentially efficacious non-hormonal treatments for menopausal symptoms because there is limited understanding of the physiological mechanisms that underlie these symptoms.

Menopausal symptoms are clearly induced by estrogen withdrawal and more abrupt decreases in estrogen are linked with greater symptomatology.

One mechanistic pathway that has not been explored is the orexin neuronal system which is restricted to the hypothalamus. Orexins (also called hypocretins) are neuropeptides first discovered in the late 1990's. There are two forms of orexins, orexin A and orexin B (also known as hypocretin 1 and hypocretin 2, respectively), that are exclusively produced in hypothalamic neurons of the lateral hypothalamic area (LHA1). Orexin A and orexin B were initially identified as endogenous ligands for two orphan G-protein-coupled receptors, now known as orexin receptor-1 (OX₁R) and orexin receptor-2 (OX₂R). The amino acid identity between the full length human OX₁R and OX₂R sequences is 64%. OX₁R has greater affinity for orexin A than orexin B by 1 order of magnitude. In contrast, OX₂R has similar affinity for both orexin A and orexin B.

Orexins constitute a novel peptide family with no significant structural similarities to known families of regulatory peptides. Orexin A is a 33-amino acid peptide of 3562 Da with two sets of intrachain disulfide bonds. It has an N-terminal pyroglutamyl residue and C-terminal amidation. The primary structure of orexin A predicted from the cDNA sequences is completely conserved among several mammalian species (human, rat, mouse, cow, sheep, dog, and pig). On the other hand, rat orexin B is a 28-amino acid, C-terminally amidated linear peptide of 2937 Da that is 46% (13/28) identical in sequence to orexin A. The C-terminal half of orexin B is very similar to that of orexin A (73%; 11/15), whereas the N-terminal half is variable. Orexin A and B are produced from a common precursor polypeptide, prepro-orexin.

Several lines of evidence suggest orexins, may be involved in menopausal symptoms. First, animal research indicates that orexin A concentrations increase as estrogen declines. In female rats, orexin expression in the hypothalamus is dramatically altered over the estrus cycle. Orexin A is highest when estrogen is lowest (i.e., proestrus [menses] in rats; Porkka-Heiskanen et al, Eur J Endocrinol. 2004 May; 150(5):737-42). Similarly when systemic estrogens are administered, there is a resulting decrease in orexin A within the hypothalamus as well as at postsynaptic target sites within the CNS (Russell et al, J Neuroendocrinol. 2001 June; 13(6):561-6). In rats, females have significantly higher hypothalamic expression of orexin precursor mRNA than males (Johren et al Endocrinology. 2002; 142:3324-3331). In addition, orexin A is an activating neuropeptide that increases heart rate, raises core body temperature, and increases energy expenditure. Increased heart rate, core body temperature, and energy expenditure occur with hot flashes in menopausal women and breast cancer survivors. Furthermore, because orexin A is an activating peptide, it promotes wakefulness. Waking episodes are a common cause of sleep disturbance in menopausal women. Rodents who receive central injections of orexin A exhibit increased wakefulness and sleep disturbance. In addition, higher orexin A concentrations in cerebral spinal fluid have been demonstrated in individuals with poor sleep quality related to restless legs syndrome, a condition that is treatable with gabapentin, a drug also used to treat hot flashes. In contrast, low concentrations of orexin A (and orexin B) and underexpression of orexin receptors in the hypothalamus have been found at autopsy in narcoleptic patients, a group who suffers from excessive daytime sleepiness (Sakurai et al, Curr Opin Clin Nutr Metab Care (2003) 6(4):353-60). Orexins have also been reported to stimulate corticosteroid production (Samson et al, Regul Pept. 2002 Mar. 15; 104(1-3):97-103), and higher cortisol concentrations are related to greater hot flash symptomatology in menopausal women (Woods et al., Menopause 2006, 13(2):212-21).

Previous studies have demonstrated that orexin projects to and excites brainstem serotonergic systems that are heavily implicated in thermoregulaton, arousal, anxiety and depression. This suggests that the deficiency of serotonin implicated in depression and anxiety might be associated with a deficiency of orexinergic neurons which may cause a negative feedback to increase orexin levels. Consistent with this, orexin concentrations in cerebral spinal fluid are higher in patients that attempted suicide the previous year and altered hypothalamic orexin systems are also implicated in animal models of depression.

Consistent with these observations applicants have proposed that orexin is hyperactive during peri-post menopausal periods (from dramatic loss of estrogen) which can lead to associated adverse symptoms clusters such as hot flashes and anxiety/depression. This makes an orexin antagonist a potential treatment option for treating symptoms associated with low estrogen concentrations in women, including for example, peri-post menopausal periods, premenstrual syndrome and postpartum.

As disclosed herein, a method is provided for treating menopausal symptoms, in women experiencing low estrogen concentrations and/or elevated orexin activity. The method comprises the step of administering an orexin inhibitor to a patient suffering from menopausal symptoms. Menopausal symptoms may result not only from dramatic loss of estrogen during natural menopause, but also frequently occurs following oophorectomy, postpartum and pre-menstruation, or following breast, ovarian cancer or endometriosis treatments that pharmacologically inhibit synthesis of estrogens and/or block the effects of estrogens at the receptor.

In regards to breast cancer (BC), it is the most common malignancy in women, and in 2005 over 2.3 million BC survivors were estimated to be alive in the United States (American Cancer Society, 2007). Breast cancer can be divided into 2 types. One subtype is estrogen receptor-positive BC (ER+BC), which depends on hormonal estrogens to grow, and accounts for about 65% of all BC patients. In ER+BC patients, the standard of care therapy includes the inhibition of estrogen action by either endocrine drug therapy (e.g. antiestrogens or aromatase inhibitors) or surgical oophorectomy. Although these therapies dramatically improve survival rates (>5-year survival rate in over 95% ER+BC patients), they also cause a cluster of adverse menopausal-associated symptoms such as anxiety, cutaneous vasomotor/sudomotor “hot flashes”, sleep disturbances, and appetite change. Among these symptoms, hot flashes are the cardinal symptom following estrogen inhibition and are experienced by 65% of ER+BC patients, with about 50% rating the symptom as severe or extremely bothersome.

Overall, adverse menopausal symptoms resulting from estrogen inhibition dramatically reduce the quality of life of ER+BC survivors and also lead to non-compliance with endocrine therapies. Treating these symptoms is important because they are a common reason for women discontinuing these life-saving endocrine therapies; by 2 years into a 5 year treatment schedule, nearly 50% of women stop taking their the antiestrogens and aromatase inhibitors. In those that do continue taking them, they continue to cause a substantial reduction in their quality of life. Unfortunately, the existing nonhormonal treatments for these symptoms are not very effective.

Accordingly there is a need for a nonhormonal treatment to provide relief to women suffering from menopausal symptoms. As disclosed herein applicants have discovered that inhibition of orexin activity can prevent or alleviate premenstrual symptoms. In one embodiment the method is administered to breast cancer patients that are receiving aromatase inhibitors. Menopausal symptoms can be exacerbated or induced by aromatase inhibitors, and therefore these women represent one group particularly suited for treatment in accordance with the methods disclosed herein

### SUMMARY

As disclosed herein menopausal symptoms are believed to cluster due to a common shared etiology related to the orexin neuronal system. Orexins (hypocretins) are activating neuropeptide hormones and hypothalamic orexin A expression is inversely related to estrogen levels (higher orexin when estrogen is lower). Applicants anticipate that women characterized by low estrogen activity and/or elevated orexin activity can be treated to relieve any associated menopausal symptoms by administration of an orexin inhibitor.

In one embodiment a method of identifying compounds that are active in treating menopausal symptoms is provided. The method comprises using an animal model of menopause (e.g. a bilateral ovariectomized rat) and administering a menopause challenge both in the presence and absence of a test compound to see if the test compound will blunt an increase in tail skin temperature induced in the menopausal model animal by the menopause challenge.

In another embodiment a method is provided for treating menopausal symptoms associated with a dramatic loss of estrogen activity that results from either reduced estrogen concentrations or from inhibition of estrogen receptor activity (e.g. resulting from aromatase inhibitory therapy). Orexin is believed to be hyperactive during peri-post menopausal periods (including surgery induced menopause) which can lead to associated adverse symptoms clusters such as hot flashes and anxiety/depression. Accordingly, a method is provided for treating any condition that can also lead to these symptoms, including premenstrual syndrome and postpartum, where estrogens would also be very low and orexin activity high.

In one embodiment the method of relieving menopausal symptoms comprises the step of administering to a patient, exhibiting below average estrogen activity and/or above average orexin activity, a composition comprising an inhibitor of orexin activity. The composition can be administered prophylactic ally, or can be administered after the onset of the symptoms. In one embodiment the method is used to treat a breast cancer or ovarian cancer patient that is receiving aromatase inhibitor therapy, to prevent the onset, or reduce the severity, frequency or duration of menopausal symptoms.

Drugs that inhibit estrogen synthesis (e.g., aromatase inhibitors), or block the effects of estrogen at the receptor (e.g., tamoxifen) are used to block the cancer promoting effects of estrogens in women with breast and ovarian cancer, but also in conditions such as endometriosis. These treatments also lead to adverse menopausal-like symptoms clusters. Accordingly, patients receiving treatments that decrease estrogen receptor activity either by reducing the synthesis of estrogen or by blocking receptor activity (e.g., administration of aromatase inhibitors or tamoxifen) will also benefit from the administration of an orexin inhibitor to prevent or alleviate menopausal symptoms.

Inhibitors of orexin activity are known to those skilled in the art and include the use of interfering RNA, anti-sense nucleic acids and peptide or other small molecules. In one embodiment the inhibitor is an orexin A receptor specific inhibitor. In another embodiment the inhibitor is an orexin B receptor specific inhibitor. Alternatively, the method may comprise the administration of both an orexin A and an orexin B receptor specific inhibitor, including for example a dual orexin A and B receptor inhibitor. In one embodiment the method comprises the administration of one or more orexin inhibitors selected from the group consisting of SB334867, MK4305 and Almorexant.

### BRIEF DESCRIPTION OF THE DRAWINGS

FIG. 1 presents data from an experiment where a panic prone rat model system is used to test the ability of an orexin 1 receptor (ORX1) antagonist SB334867 to ameliorate the panic response. The rats were initially made panic prone by a chronic infusion of a GABA synthesis inhibitor: 1-allylglycine [(1-AG; directed at orexin neurons that are exclusive to the dorsomedial and lateral hypothalamus (DMH and LH) (Peyron et al., J Neurosci 1998, 18:9996-10015): see orexin immunoreactive neurons in FIG. 1a]. The panic prone mice were then systemically injected with either the orexin 1 receptor (ORX1) antagonist [SB334867, 30 mg/kg i.p., Tocris Bioscience, Bristol, UK, in 0.2 ml/100 g volume DMSO, i.p.] or benzodiazepine (alprazolam, 3 mg/kg i.p., Sigma), prior to an ordinarily mild interoceptive stressor (i.e., 15 min i.v. infusion of 0.5M sodium lactate challenge). Administration of the orexin 1 receptor (ORX1) antagonist attenuated b) “anxiety”-like responses [social interaction (SI) duration; n=14,8,11,12,6; treatment effect F(4,36)=17.8, p=0.001) and lactate induced increases in c) core body temperature (n=6/group, treatment×time effect F(14,98)=1.9, p<0.04), d) heart rate (HR, n=6/group; treatment×time effect F(14,98)=5.4, p<0.001) and e) general locomotor activity (n=6/group; treatment×time effect F(14,98)=1.8, p<0.05), * indicate significant differences between Vehicle treated groups and SB33 and alprazolam group using Fisher's LSD poshoc tests that are protected with an ANOVA at each time point with p<0.05. Mean values of baseline temperature, HR and activity were not significantly different.

FIG. 2 Continuous and simultaneous measurement of blood pressure (top panel), heart rate (middle panel), and core body temperature (bottom panel) in an isoflurane anesthetized rat after identical microinjections of BMI (20 pmol/50 nL) into the DMH (time marks 1 and 3) before and after systemic administration of the orexin-1 receptor antagonist SB334861 (30 mg/kg i.p.; time mark 2). Note attenuation of all three measures after the administration of the orexin receptor antagonist.

FIG. 3A & 3B are bar graphs showing the effects of subcutaneously injecting adult female rats with 3 days of vehicles (10% DMSO, 90% sesame oil) or 10 mg/kg tamaoxifen (a nonselective estrogen receptor antagonist) on time spent in the center of an open field. * represents p,0.05 with unpaired t-test. n=6/group. Rats were also injected intraperitoneally with an orexin 1 receptor antagonist (SB334867, 30 mg/kg) into ovariectomized (OVEX) female rats 30 min prior to anxiety testing. The data in FIG. 3B indicates that the orexin inhibitor reverses anxiogenic effects (reduced time spent in center of an open field box) of menopausal state (n=10/group). Bars represent mean and error bars represent SEM. * represents p,0.05 Mann Whitney U test following an ANOVA f(3,35)=2.9, p=0.048.

FIG. 4A-4C are graphs demonstrating the effects of ovariectomy (OVEX, n=4) verses sham-OVEX surgeries (n=4) on core body temperature (FIG. 4A), tail temperature (FIG. 4B) and locomotor activity (FIG. 4C) over a 24 hour period. Lines represent mean +/−SEM. The dark bar on the x-axis represents the night cycle (active phase).

FIG. 5A & 5B are graphs showing the effects of yohimbine on cellular responses. FIG. 5A shows the effect of an intraperitoneal injection of Yohimbine (Sigma; 5 mg/kg: n=6) or saline vehicle on cellular responses (c-Fos induction) in ORX-ir neurons of male rats. Black and gray outlined bars respectively represent c-FOS/ORX double and total ORX-ir labeled neurons in the hypothalamus. *Mann-whiney U=1.5, p=0.001. FIG. 5B shows the effects of yohimbine (5 mg/kg, i.p.) on tail skin temperature in female ovariectomized (n=2), or sham-OVEX control (n=3), rats. Treatment by time effect F(89,267)=2.7, p<0.001.

FIG. 6A & 6B present data on the effects of hypercarbic gas exposure on cellular responses. FIG. 6A shows the effect of 5 minute hypercarbic (20% CO₂) gas exposure on cellular responses (cFos induction) in ORX-immunoreactive neurons of male rats (n=7/group). Black and Gray outlined bars respectively represent c-FOS/ORX double and total ORX-ir labeled neurons in the hypothalamus, *F(1,12)=11.2, P=0.0006, unpaired t-test. FIG. 6B shows the effects of 20% CO₂ on tail skin temperature in female ovariectomized, or sham-OVEX control, rats systematically pretreated with vehicle or an ORX1 receptor antagonist (SB334867, 30 mg/kg).

FIG. 7 presents data on the effects of a brief (5 minute) hypercarbic (20% CO₂) gas exposure on cellular responses (c-Fos induction) in ORX-immunoreactive neurons of male rats (n=7/group). FIG. 7A: Black and gray outlined bars respectively represent c-FOS/ORX double and total ORX-ir labelled neurons in the hypothalamus, *F(1,12)=11.2, P=0.006, unpaired t-test. FIG. 7B: is a graph showing the effects of 20% CO₂ on tail skin temperature in female ovariectomized, or sham-OVEX control, rats systemically pretreated with vehicle or an ORX1 receptor antagonist (SB334867, 30 mg/kg). There was an Orexin 1 receptor antagonist×OVEX×time effect [F(12, 126)=2.3, p=0.007]. *,# and + symbols denote significant differences between groups using a Tukey's HSD posthoc test p<0.05.

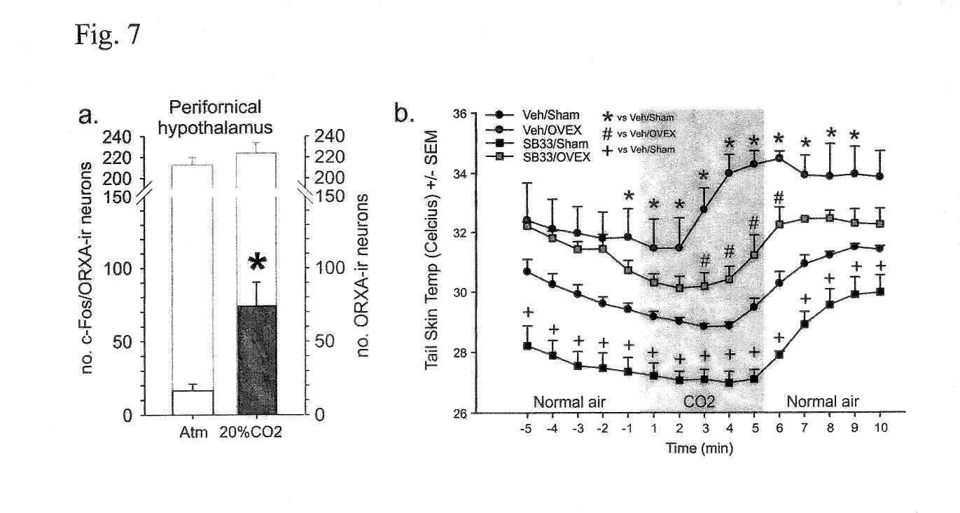

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable.

As used herein, the term “treating” includes prophylaxis of the specific disorder or condition, or alleviation of the symptoms associated with a specific disorder or condition and/or preventing or eliminating said symptoms.

As used herein the term “inhibitor of orexin activity” or an “orexin inhibitor” is intended to encompass any safe and effective non-hormonal compound or treatment that can be administered to a patient to decrease orexin activity in vivo. Orexin activity includes binding of an orexin (i.e., orexin A or orexin B) to one of its corresponding G-protein coupled orexin receptors, ORX1 and ORX2, and activation of signal transduction pathways. As used herein, the term “menopausal symptoms” refers to any undesirable symptom that is typically associated with the progression of a women through menopause, or associated with any other condition or treatment that causes a significant reduction in estrogen activity. Examples of menopausal symptoms include hot flashes, night sweats, irregular periods, loss of libido, mood swings, fatigue, sleep disturbance and vaginal dryness.

As used herein, the term “menopausal model” relates to any female animal that has received a treatment that makes the animal susceptible to menopausal symptoms, typically by substantially reducing estrogen activity. One example of a menopausal model would be a bilaterally ovariectomized rodent.

“Estrogen Receptor” as defined herein refers to any protein in the nuclear receptor gene family that binds estrogen, including, but not limited to, any isoforms or mutations having the characteristics just described. More particularly, the present invention relates to estrogen receptor(s) for human and non-human mammals (e.g. animals of veterinary interest such as horses, cows, sheep, and pigs, as well as household pets such as cats and dogs). Human estrogen receptors include the alpha- and beta-isoforms (referred to herein as “ERalpha” and “ERbeta”).

As used herein the term “estrogen activity” refers to the binding of estrogen to its corresponding receptor and activation of signal transduction pathways. An individual characterized with having reduced estrogen activity is one who has either lower concentrations of estrogen, or a reduced ability of estrogen to bind and/or activate the estrogen receptor signal transduction pathways, relative to healthy individuals of the same age.

“Hot flashes” refer to events impacting blood vessel diameter and characterized by the sudden onset of intense warmth that may begin in the chest and may progress to the neck and face. They are often accompanied with palpitations, profuse sweating, and red blotching of the skin.

As used herein an “effective” amount or a “therapeutically effective amount” of an orexin inhibitor refers to a nontoxic but sufficient amount of an inhibitor to provide the desired effect. For example one desired effect would be preventing the onset, or reducing the severity, frequency or duration of menopausal symptoms. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “patient” without further designation is intended to encompass any warm blooded vertebrate domesticated animal (including for example, but not limited to livestock, horses, cats, dogs and other pets) and humans.

As used herein the term “cancer patient” is intended to encompass any patient that at one time was diagnosed with cancer and continues to receive treatments related to their cancer. This includes patients with active cancers, those in remission, and patients who have been subsequently deemed cancer free but continue to receive treatment for their cancer. For example breast cancer or ovarian cancer patients may continue to receive aromatase therapy for their cancers long after cancer cells can no longer be detected in their bodies.

### Embodiments

Menopause is a condition where estrogen levels are severely depleted which leads to a cluster of adverse symptoms such as anxiety, vasomotor/sudomotor “hot flash” or “night sweats”, sleep disturbances, depression, and appetite change. Currently, estrogen therapy (ET) is the first line treatment for menopausal symptoms. However, estrogen therapy is no longer acceptable because of shifts in its risk (e.g., cancer)-benefit ratio. Therefore, there is a need for either more specific estrogen agonists that reduce adverse menopausal symptoms without inducing tumoregenic effects; and/or non-hormonal therapies to reduce the incidence of adverse menopausal symptoms. Unfortunately, the scientific understanding of menopausal symptoms is limited, and the few non-hormonal therapies that exist are much less effective than ET and have adverse side effects.

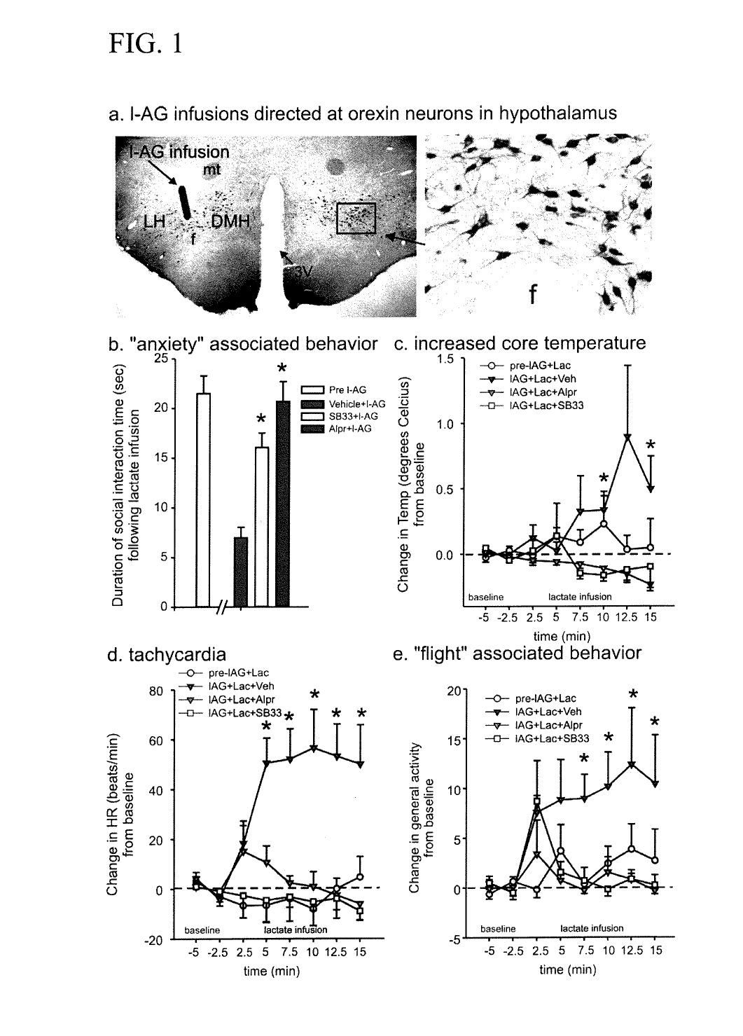

As disclosed herein a non-hormonal method is provided to prevent or treat adverse peri- or post-menopausal symptoms. Orexin (also known as hypocretin) synthesizing neurons, which are exclusive to the perifornical hypothalamic region (PeF), play a critical role in wake-promotion; vasomotor and thermogenic mobilization and appetite, all of which are all components of menopausal symptoms. In a recent Nature Medicine article (Johnson et al., 2010 16(1): p. 111-5), applicants demonstrated that orexin neurons are hyperactive in anxious rats and central orexin levels are elevated in patients with anxiety symptoms. In addition, anxiety and panic associated responses could be blocked by either silencing the hypothalamic orexin gene or by systemic treatment with an orexin antagonist (See Example 1 and FIG. 1). This suggests that the orexin system may be involved in the patho-physiology of anxiety states, and that orexin antagonists constitute a potential novel treatment strategy for anxiety related conditions.

Recent animal and human research indicates that central orexin activity is inversely correlated with peripheral estrogen levels. In female rats, orexin expression in the hypothalamus is highest when estrogen levels are low (Porkka-Heiskanen et al., Eur J Endocrinol (2004) 150:737-742), and systemic estrogen administration decreases orexin A within the hypothalamus and at CNS postsynaptic target sites (Russell et al., Endocrinology (2001) 142(12):5294-302). Finally, a recent human study showed that compared to reproductive female controls, menopausal women had 300% higher orexin levels in their cerebrospinal fluid, that were restored to control levels following estrogen therapy.

The present disclosed methods are based on the premise that the orexin system constitutes a key mechanism for promoting wakefulness and mobilizing an integrative stress responses (behavioral and autonomic) and that menopause and breast cancer endocrine therapies induce menopausal symptoms (e.g., anxiety, sleep disruption, vasomotor/thermogenic hot flashes) by altering the normal estrogenic inhibitory control of the orexin system. Applicants recognized that an orexin antagonist is a potential option for treating symptoms associated with low estrogen concentrations in women, including for example, peri- post menopausal periods, premenstrual syndrome and postpartum or surgical ovariectomy (oophorectomy). Accordingly, in one embodiment, menopausal symptoms are expected to be treatable or preventable by either using selective estrogen receptor β (ERβ) agonist therapies that do not impact the orexin system; or through the use of inhibitors of orexin activity, including for example, ORX 1 or 2 receptor antagonists.

In accordance with one embodiment a method of treating menopausal symptoms is provided. The method comprises reducing orexin activity in a patient suffering from one or more menopausal symptoms including, hot flashes, night sweats, irregular periods, loss of libido, vaginal dryness, mood swings, fatigue, hair loss, sleep disorders, difficult concentrating, memory lapses, dizziness, weight gain, incontinence, bloating, allergies, brittle nails, changes in odor, irregular heartbeat, depression, anxiety, irritability, panic disorder, breast pain, headaches, joint pain, burning tongue, electric shocks, digestive problems, gum problems, muscle tension, itchy skin, tingling extremities and osteoporosis. The most common menopausal symptoms include hot flashes, night sweats, irregular periods, loss of libido, vaginal dryness and mood swings, and in accordance with one embodiment a method of treating one of those six symptoms is provided. In a further embodiment a method of treating hot flashes associated with women with low estrogen levels is provided wherein the activity of orexins is reduced.

In one embodiment a method of treating a female patient for menopausal symptoms comprises a step of first identifying female patients that have below average estrogen activity and/or above average orexin activity. Patients suffering from relatively low estrogen activity (e.g., either low estrogen concentrations or low estrogen receptor activation) are then administered a composition comprising an inhibitor of orexin activity, in an amount sufficient to prevent the onset, or reduce the severity, frequency or duration of menopausal symptoms. In one embodiment the orexin inhibitor is an ORX1, ORX2 or dual ORX1/ORX2 receptor antagonist. The orexin inhibitors can be administered alone or in conjunction with other known compositions and treatments to enhance the effectiveness of the known treatments in treating menopausal symptoms. In accordance with one embodiment the orexin inhibitor is administered in conjunction with the administration of a nonsteroidal anti-inflammatory drug (NSAID) such as ibuprofin. Advantageously, when the orexin inhibitor is administered in conjunction with other agents used for treating menopause, the amount of the active agents (including the orexin inhibitor) needed for efficacy may be reduced relative to when one agent is used alone. For example, orexin inhibitors can be co-administered with estrogen or an estrogen receptor agonist as a means of reducing the amount of estrogen or estrogen receptor agonist needed to prevent or reduce the severity, frequency or duration of said menopausal symptoms.

The term “co-administered” as used herein means that the second therapeutic agent may be administered together with an orexin inhibitor as part of a single dosage form or as separate, multiple dosage forms. Alternatively, the additional agent may be administered prior to, consecutively with, or following the administration of the orexin inhibitor, however the two therapeutic agents are administered within a timeframe wherein the first therapeutic agent is still active in vivo upon administration of the second therapeutic agent or treatment. The co-administration of an orexin inhibitor and a second therapeutic agent, to a patient does not preclude the separate administration of that same therapeutic agent, any other second therapeutic agent or any compound of this invention to the patient at another time during a course of treatment.

In accordance with one embodiment an inhibitor of orexin activity is administered to a woman to prevent or reduce the severity, frequency or duration of hot flashes. The orexin inhibitor can be administered either after the first onset of menopausal symptoms or the orexin inhibitor can be administered prophylaticly based on the patient exhibiting below average estrogen activity and/or above average orexin activity.

Drugs that inhibit estrogen synthesis (e.g., aromatase inhibitors), or block the effects of estrogen at the receptor (e.g., tamoxifen) are used to block the cancer promoting effects of estrogens in women with breast and ovarian cancer, but also in conditions such as endometriosis. These treatments also lead to adverse menopausal-like symptoms clusters. Accordingly, patients receiving treatments that decrease estrogen receptor activity either by reducing the synthesis of estrogen or by blocking receptor activity (e.g., administration of aromatase inhibitors or tamoxifen) will also benefit from the administration of an orexin inhibitor to prevent or alleviate menopausal symptoms.

In accordance with one embodiment, a patient receiving drugs that either inhibit estrogen synthesis (e.g., aromatase inhibitors), or block the effects of estrogen at the receptor (e.g., tamoxifen), are co-administered with an inhibitor of orexin activity as a means of preventing the onset, or reduce the severity, frequency or duration of menopausal symptoms in such patient. In one embodiment the patient is a breast cancer or ovarian cancer patient receiving aromatase inhibitor therapy or other drugs that block the effects of estrogen at the receptor. In one embodiment the orexin inhibitor is administered prior to or simultaneously with the administration of aromatase inhibitor or the estrogen receptor blocking drug.

Reducing orexin activity can be accomplished by interfering with the expression of orexin A and/or orexin B through standard techniques known to those skilled in the art including for example the use of a short hairpin RNA (shRNA), microRNA, antisense molecule, a small double stranded interference RNA (siRNA) directed to at least one of the genes that codes for orexin. Alternatively, the activity of orexins can be reduced by an antibody or other molecule that binds to orexin A and/or orexin B or their receptors, or otherwise interferes with the interaction of orexin A and/or orexin B with one or more of the ORX 1 or 2 receptors. As used herein the term “inhibitor of orexin activity” is intended to encompass any safe and effective nonhormonal compound or treatment that can be administered to a patient to inhibit orexin activity in vivo. In accordance with one embodiment the inhibitor of orexin activity is an ORX 1 or 2 receptor antagonist. Preferably the compound is one that can penetrate the blood brain barrier. Orexin receptor antagonists having these properties are known to those skilled in the art and include, for example, inhibitors SB334867 (1-(2-methylbenzoxazol-6-yl)-3-[1,5]naphthyridin-4-yl urea):

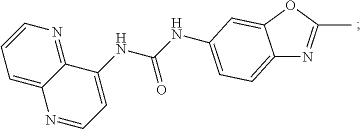 [SMILES: Cc1nc2ccc(NC(=O)Nc3ccnc4cccnc34)cc2o1]

MK4305 (a proprietary compound of Merck currently in Phase III testing; see Baxter et al, Org. Process Res. Dev., 2011, 15 (2), pp 367-375, the disclosure of which is incorporated herein by reference):

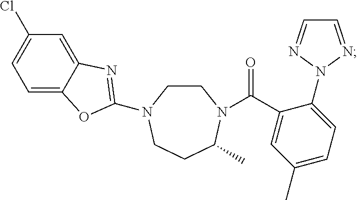 [SMILES: Cc1ccc(-n2nccn2)c(C(=O)N2CCN(c3nc4cc(Cl)ccc4o3)CC[C@H]2C)c1]

and Almorexant (2R)-2-[1S)-6,7-dimethoxy-1-{2-[4-(trifluoromethyl)phenyl]ethyl}-3,4-dihydroisoquinolin-2(1H)-yl]-N-methyl-2-phenylacetamide):

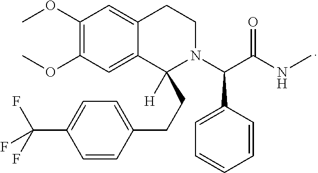 [SMILES: CNC(=O)[C@@H](c1ccccc1)N1CCc2cc(OC)c(OC)cc2[C@@H]1CCc1ccc(C(F)(F)F)cc1]

SB334867 was the first non-peptide antagonist developed that is selective for the orexin receptor subtype ORX1, with around 100× selectivity for ORX1 over ORX2 receptors. Both MK4305 and Almorexant are competitive, dual ORX₁ and ORX₂ receptor antagonists and selectively inhibit the functional consequences of ORX₁ and ORX₂ receptor activation. In accordance with one embodiment, one or more of these inhibitors are administered to a patient, having below average estrogen activity and/or above average orexin activity, in an amount sufficient to prevent the onset, or reduce the severity, frequency or duration of menopausal symptoms, including hot flashes, night sweats, irregular periods, loss of libido, vaginal dryness and mood swings. In one embodiment the orexin inhibitor is administered at a dosage of about 5 mg/kg to about 100 mg/kg, about 12 mg/kg to about 80 mg/kg, about 12 mg/kg to about 60 mg/kg, about 20 mg/kg to about 50 mg/kg, about 20 mg/kg to about 40 mg/kg, about 24 mg/kg to about 36 mg/kg or about 27 mg/kg to about 33 mg/kg.

In accordance with one embodiment a pharmaceutical composition is prepared comprising an orexin inhibitor and a pharmaceutically acceptable carrier. In one embodiment an orexin inhibitor is used in the manufacture of a medicament for the treatment of menopausal symptoms. Thus in accordance with one embodiment an orexin inhibitor, including for example an orexin receptor antagonist, is used to treat menopausal symptoms, including for example, hot flashes. In one embodiment the pharmaceutical composition comprises an orexin inhibitor selected from the group consisting of SB334867, MK4305 and Almorexant and a pharmaceutically acceptable carrier.

In one embodiment, a patient is administered a composition comprising the orexin inhibitor in a standard pharmaceutically acceptable carrier using any of the standard routes of administration known those skilled in the art. The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the orexin inhibitor is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. The pharmaceutical compositions of this invention may be administered in the form of suppositories for rectal or vaginal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation. In one embodiment the composition is administered by injection, and more particularly, by intravenous or subcutaneous injection.

In accordance with one embodiment the patient treated with the orexin inhibitory composition is a breast cancer survivor that is also receiving aromatase inhibitor therapy. The breast cancer survivor can be administered the orexin inhibitory composition in conjunction with the aromatase inhibitor therapy, with the orexin inhibitory composition being administered either before and/or during the time of the aromatase inhibitor therapy. In one embodiment the orexin inhibitory composition is co-administered with the aromatase inhibitor either as a single composition or as two separate compositions administered within one to two hours of each other. In accordance with one embodiment a composition is provided comprising an aromatase inhibitor and an orexin inhibitor. In one embodiment the aromatase inhibitor is anastrozole or letrozole and the orexin inhibitor is selected from the group consisting of SB334867, MK4305 and Almorexant.

In accordance with one embodiment a method is provided for identifying candidate compounds that will alleviate menopause symptoms, including for example hot flashes. The method uses female mammalian species, including rodents such as rats and mice, that are treated to dramatically reduce their endogenous estrogen levels and thus make them susceptible to menopause symptoms. Specifically, applicants have found that bilaterally ovariectomized (OVEX) or pharmacological estrogen blocked rats can serve as a valid rat model of multiple adverse menopausal-related symptoms. Accordingly, the skin and/or core body temperatures of animals modified to have suddenly reduced estrogen levels (e.g., OVEX rats) are monitored over time, with or without administration of a test compound, to determine if the test compound can reduce increases in temperature, including for example increased tail temperature, during the animal's active phase. Core body temperature can be monitored in the model animal to see if there is a dramatic drop in core body temperature following the increased temperature in the tale, as a validation of a hot flash, since such a core body temperature drop also occurs in women following the onset of a hot flash. As used herein the active phase of an animal refers to an animal's wakeful period during a 24 hour cycle and includes when the animal is physical activity. In accordance with one embodiment an average increase in temperature, or failure to reduce the average temperature, of the tail skin during the active phase will be considered an indication of the animals susceptibility to menopausal symptoms. A test compound that can reverse elevated active phase tail skin temperatures observed with the menopausal model animals will identify candidate compounds for treating women suffering from menopausal symptoms. Furthermore, in one embodiment, a test compound's ability to induce a reduction in a menopausal model animal's tail temperature as the animal enters it active phase will identify candidate compounds. In one embodiment, monitoring of activity and core body and/or skin temperature will occur 1 hr before onset of the inactive sleep cycle and stop 1 hr post active phase. In one embodiment the selection of a test compound as a candidate compound for alleviating menopause symptoms will be based on whether the skin temperature of the menopausal model animal's tail is maintained or reduced as the animal enters into its active phase.

In accordance with one embodiment a method for identifying candidate compounds for activity in relieving menopausal conditions is provided. In one embodiment the menopausal condition is hot flash symptoms associated with low estrogen levels in female patients. In one embodiment the method comprises

a) providing a freely moving, non-anesthetized female rodent menopausal model (typically a female rat);

b) administering a test compound to said rodent menopausal model;

c) administering a menopausal challenge to said rodent menopausal model; and

monitoring the core body temperature and/or tail temperature of said rodent menopausal model during the administration of the menopause challenge to identify test compounds that prevent an increase of greater than 1° C. from baseline temperature as candidate compounds. The menopausal model can be any female animal that has been subjected to treatment to causes a sudden reduction in estrogen activity in the animal. Such treatments include, ovariectomization, administration of pharmaceuticals that either interfere with estrogen synthesis, stability or its ability to activate its receptor. In one embodiment the menopausal model is a bilaterally ovariectomized adult female rodent, and in one embodiment an ovariectomized female rat. Advantageously, the present method can be conducted on freely moving non-anesthetized animals.

The menopausal challenge can be selected from any treatment that is known to induce menopausal symptoms in individuals susceptible to said symptoms. Such treatments include for example, administration of pharmaceutical agents such as yohimbine, subjecting the test subject to hypercapnia conditions (e.g. exposure to elevated CO₂ levels such as 20% CO₂) or elevated ambient temperatures. Those administered test compounds that are capable of blunting the response of the menopausal model when exposed to the menopause challenge are identified as candidate compounds that have utility for treating women suffering from menopausal symptoms. In accordance with one embodiment candidate compounds will be identified as those test compounds that prevent an increase in temperature (either core body temperature and/or tail skin temperature) of no more than 2.0° C., 1.5° C., 1.0° C., 0.5° C. from the baseline temperature upon administration of the challenge compound.

Typically the temperature of the animal is monitored during and after the administration of the challenge, including for example 1 hour prior to administration of the challenge, throughout the time the challenge is administered, and for hour after administration of the challenge is ended. However in one embodiment the animal is monitored for a 24 hour period. In one embodiment the animal model system is an OVEX or pharmacological estrogen blocked rodent, including for example a rat. In accordance with one embodiment the challenge comprises administration of yohimbine or a hypercapnic gas. In one embodiment the test compound is identified as a suitable candidate if the test compound causes a delay or reduced intensity in tail skin temperature of the model animal after administration of the challenge relative to the model animal that is administered the challenge in the absence of the test compound.

In accordance with one embodiment 24 hr tail skin and core body temp and sleep-related locomotor activity will be assessed by using radio-telemetry probes; followed by anxiety behavior testing. In one embodiment test compounds will be injected into the bloodstream of the model animal to investigate whether the compound can attenuate menopausal-associated activity induced by “hot flash” provocation (assessed by tail and core body temp) following yohimbine or hypercapnia challenge.

In one embodiment a kit is provided for administering the orexin inhibitor to a patient. The kit comprises one or more orexin inhibitors and a device for administering the orexin inhibitor to a patient. Depending on the route of administration, the kit may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit. In one embodiment the kit is provided with a device for administering an orexin inhibitor composition to a patient, e.g. syringe needle, pen device, jet injector or other needle-free injector. In one embodiment the device is a syringe. In one embodiment the kit further comprises reagents for administering a menopausal challenge.

The kit may alternatively or in addition include one or more containers, e.g., vials, tubes, bottles, single or multi-chambered pre-filled syringes, cartridges, infusion pumps (external or implantable), jet injectors, pre-filled pen devices and the like, optionally containing the orexin inhibitor in a lyophilized form or in an aqueous solution. Preferably, the kits will also include instructions for use. In one embodiment, the kits of this invention may comprise in a separate container a pharmaceutical composition comprising a second therapeutic agent, for co-administration with an orexin inhibitor. In one embodiment the orexin inhibitor is selected from the group consisting of SB334867, MK4305 and Almorexant. In a further embodiment a kit for preventing menopausal symptoms in a breast cancer survivor that is also receiving aromatase inhibitor therapy is provided. In this embodiment the kit further comprises an aromatase inhibitor, including for example, anastrozole or letrozole.

In accordance with one embodiment a method of treating menopausal symptoms is provided wherein the patient is administered a composition that decreases overall orexin receptor activity (i.e., activity produced by both the ORX1 and ORX1 receptors). In another embodiment the method of treating menopausal symptoms is provided wherein the patient is administered a composition that selectively decreases orexin receptor activity of the ORX1 or ORX2 receptor. In accordance with one embodiment a method of treating menopausal symptoms is provided wherein the patient is administered an orexin inhibitory compound that is a selective orexin receptor subtype ORX1 antagonist. In an alternative embodiment a method of treating menopausal symptoms is provided wherein the patient is administered an orexin inhibitory compound that is selective for the orexin receptor subtype ORX2. In a further embodiment the patient is administered a composition that comprises a dual ORX₁ and ORX₂ receptor antagonists. In one embodiment the patient is administered a selective inhibitor of orexin A activity. In an alternative embodiment the patient is administered a selective inhibitor of orexin B activity. In another embodiment the patient is co-administered a selective inhibitor of orexin A activity and second orexin inhibitor wherein the second orexin inhibitor is either a dual ORX₁ and ORX₂ receptor antagonist or a selective inhibitor of orexin B activity.

In accordance with one embodiment a method of preventing the onset, or reduce the severity, frequency or duration of menopausal symptoms in a breast cancer survivor receiving aromatase inhibitor therapy is provided. The method comprises administering to said breast cancer survivor a composition comprising an inhibitor of orexin activity. In one embodiment the inhibitor of orexin activity comprises an orexin receptor antagonist, including one or more of the compounds SB334867, MK4305 and Almorexant. In one embodiment the inhibitor is SB334867. The compounds are typically administered by intravenous injection at a dosage of about 20 mg/kg to about 50 mg/kg, more typically at about 24 mg/kg to about 36 mg/kg. In one embodiment the orexin receptor antagonist is administered at a dosage of about 27 mg/kg to about 33 mg/kg or at a dosage of about 30 mg/kg.

### EXAMPLE 1

### Administration of Orexin 1 Receptor Antagonist (SB334867) Blocks Sodium Lactate Induced Increases in Anxiety

Panic disorder is associated with disruption of central inhibitory gamma-aminobutyric acid (GABAergic) tone, and may contribute to panic attacks which are consistently provoked by ordinarily mild physical stressors such as mild osmotic disturbances (e.g., intravenous sodium lactate challenges). Similarly, chronic disruption of GABAergic tone in the dorsomedial hypothalamus (a site that is critical for regulating adaptive panic/defense responses) of rats produces a vulnerability to panic-like anxiety and flight associated behavior and cardiorespiratory responses following intravenous 0.5 M sodium lactate, (Shekharetal., Pharmacol Biochem Behav (1996) 55:249-256 and Johnson et al., Nature medicine (2010) 16:111-115. providing an animal model of panic disorder.

Orexin/hypocretin producing neurons are almost exclusive to dorsomedial hypothalamus and adjacent lateral hypothalamus, and play a critical role in maintaining wakefulness, vigilance and central sympathetic mobilization, which are key components of panic and anxiety. Experiments were conducted to investigate whether the administration of either a systemic orexin 1 antagonist or silencing the hypothalamic orexin gene product with RNA interference could block lactate-induced panic responses. The resulting data supports the hypotheses that panic-prone states in the animal model of panic are associated with selective activation of orexin-containing neurons. Furthermore, either systemic orexin 1 antagonists or silencing the hypothalamic orexin gene product with RNA interference blocked lactate-induced panic responses. Graphs in FIGS. 1b-e demonstrate that systemically injecting panic-prone rats with an orexin 1 receptor antagonist blocks sodium lactate induced increases in anxiety (FIG. 1b) and flight behavior (FIG. 1e) as well as thermogenic (FIG. 1c) and cardioexcitatory (FIG. 1d) responses. Thus, the data provides supports that aberrations of the hypothalamic orexin system underlie vulnerability to panic and potentially menopausal symptom clusters (e.g., sleep disturbance, anxiety, depression). Accordingly orexin antagonists should provide a novel approach to treating these symptoms/symptom clusters.

### EXAMPLE 2

### Orexin Mobilizes Anxiety-Like Behavior, Thermogenesis and Cardioexcitation

In the 1920's and 1940's the dorsomedial hypothalamic region (DMH) was shown to be critical for a “fight-or-flight”-related anxiety behavior and cardiovascular responses to deal with an imminent threat (Bard & Mountcastle, Res. Publ. Ass. Ment. Nerv. Dis. (1948) 27: 362-404; Hess & Brugger, Hely Physiol Pharmacol Acta, (1943) 1, 33-52). Orexin (ORX) neurons are concentrated within the DMH at the same location where chemical disinhibition increases anxiety-associated behavior heart rate, blood pressure, and core body temperature. In 2003 Kayaba and colleagues showed that increases in heart rate and blood pressure evoked by similar chemical disinhibition of the DMH are attenuated in orexin knockout mice (Kayaba et al., Am J Physiol Regul Integr Comp Physiol (2003) 285(3):R581-R93).

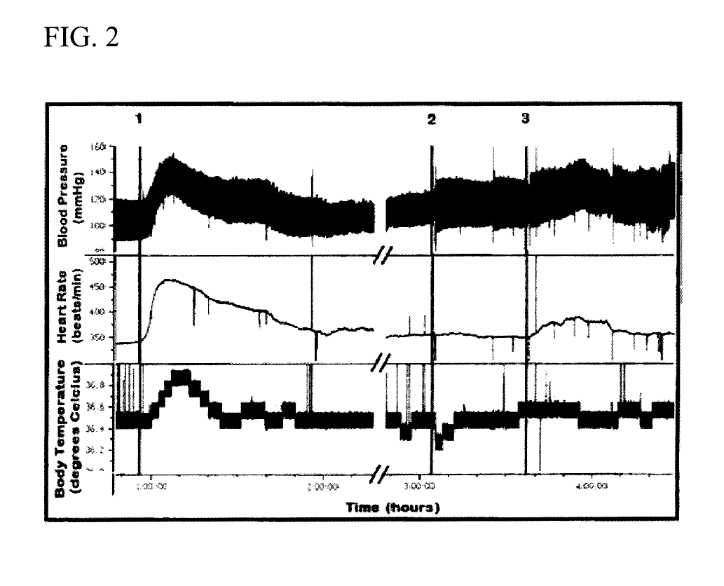

As disclosed herein, experiments were conducted to determine whether an orexin-1 receptor antagonist (SB334861) or an orexin-2 receptor antagonist (TCSOX229) could attenuate the physiologic responses to chemical stimulation of the DMH. Chemical stimulation or disinhibition of neurons in the region of the dorsomedial hypothalamus (DMH) was conducted using the GABA_{A} receptor antagonist bicuculline methiodide (BMI). To test orexin inhibitors ability to attenuate the BMI stimulated response, BMI was microinjected (20 pmol/50 nL) into the DMH of urethane-anesthetized rats both before and after systemic administration of the orexin inhibitor, and the responses were monitored. The results are shown in FIG. 2, where continuous and simultaneous measurement of blood pressure (top panel), heart rate (middle panel), and core body temperature (bottom panel) were taken in an isoflurane anesthetized rat after identical microinjections of BMI (20 pmol/50 nL) into the DMH (time marks 1 and 3) before and after systemic administration of the orexin-1 receptor antagonist SB334861 (30 mg/kg i.p.; time mark 2). Note the significant attenuation of increased heart rate, blood pressure, and core body temperature that would normally occur after microinjection of BMI. This attenuation of all three measurements is the result of administration of the orexin receptor antagonist.

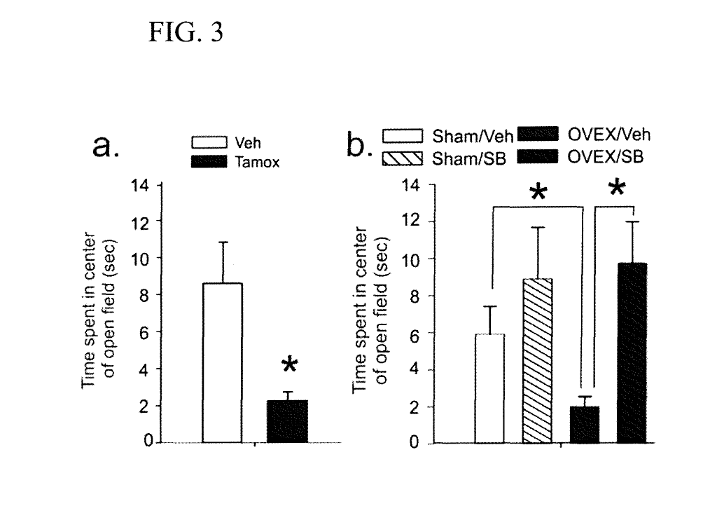

Furthermore, experiments were conducted to investigate the impact of lowering estrogen activity by either administering tamoxifen or ovariectomizing rats. The data demonstrate that tamoxifen injections (FIG. 3A), or OVEXing (FIG. 3B) female rats increased anxiety-like behavior compared to controls. Importantly, systemic injection of an ORX 1 receptor antagonist prior to conducting the analysis was found to reverse the anxiogenic effects of OVEX (FIG. 3B).

### EXAMPLE 3

### Analysis of Blood Samples from Breast Cancer Survivors Treated with Aromatase Inhibitors

Design: Blood samples and menopausal symptom data are being collected at baseline and 1, 3, 6, and 12 months after initiating therapy with the aromatase inhibitors exemestane or letrozole. The Principal Investigator of the aromatase inhibitor study has provided permission for applicants to evaluate a limited set of data from women enrolled to date. This data will consist of blood samples and symptom data from baseline prior to initiating aromatase inhibitors and one month later.

Variables: The following variables are measured at baseline and one month later. Orexin A plasma concentrations, physiological (objective) hot flashes, and subjective ratings of sleep disturbance, mood disturbance (anxiety, depressive symptoms), and vaginal symptoms (dryness, dyspareunia).

Orexin A plasma concentrations: Frozen serum samples from an earlier ELPh clinical trial will be used for biomarker association and genetics studies. We will use samples from the baseline (prior to treatment with the aromatase inhibitor) and 1 month into the therapy. Samples were collected, allowed to clot, and centrifuged to obtain the serum. They were immediately alliquoted and frozen (−80° C.). The designated samples will be obtained from the freezer and thawed on ice. The following steps will be taken for plasma orexin A extraction and radioimmunoassay: (1) thawed plasma will be acidified with an equal amount of buffer A (cat. no. RK-BA-1, Phoenix Pharm. Inc.), this will be mixed and centrifuged at 6,000 to 17,000×g for 20 min at 4° C.; (2) equilibrate a SEP-COLUMN containing 200 mg of C18 (cat. no. RK-SEPCOL-1, Phoenix Pharm. Inc.) by washing with buffer B (1 ml, once; cat. no. RK-BB-1, Phoenix Pharm. Inc.) followed by buffer A (3 ml, 3 times); (3) [NOTE: From steps 3-5, no pressure should be applied to the column] Load the acidified plasma solution onto the pre-treated C-18 SEP-COLUMN; (4) Slowly wash the column with buffer A (3 ml, twice) and discard the wash; (5) Elute the peptide slowly with buffer B (3 ml, once) and collect eluant into a polystyrene tube; (6) Evaporate eluant using a lyophilizer; (7) Dissolve the residue in RIA buffer for radioimmunoassay as follows: For a normal subject, dissolve in 250 ul RIA buffer for a two-tube assay. Aliquot 100 ul into each tube (50 ul is left over). If each tube is found to contain 10 pg of the peptide, then the total level of peptide in plasma=10 pg/tube×2.5 tubes=25 pg/ml. If upon assay the peptide value exceeds or does not fall in the range of detection, dilute or concentrate the samples accordingly; (8) Measure the test tubes for 1 min in a gamma scintillation counter (Beckman Gamma DP5500).

Measuring plasma concentrations of orexin A serves as a valid indicator of orexin neuronal system functioning for two reasons. First, in mice, orexin A crosses the blood brain barrier with ease. Second, in humans, narcolepsy (a neurological condition most characterized by excessive daytime sleepiness) is heavily linked to a dysfunctional orexin system that is evidenced by decreases in the numbers of hypothalamic orexin neurons as well as decreased concentrations of orexin A in cerebrospinal fluid and in peripheral plasma.

In addition, the time of day the blood samples were drawn should not influence measurements. Previous research has shown no circadian variability in plasma orexin A using radioimmunoas say. Baseline blood samples will be drawn at a mean time of 9:49 am (SD=1.05 hrs). One month blood samples will be drawn at a mean time of 10:55 am (SD=2.02 hrs).

Hot flashes: Objective hot flashes are measured using sternal skin conductance monitoring as described previously. (Carpenter et al., (2004) Oncol Nurs Forum 31:591-5598 (UFI, Model 7-day 3991 SCL, Morro Bay, Calif.). Participants wear the monitor for 36-hours at each assessment point. Data from the monitor are downloaded and the number of hot flashes was evaluated by trained raters with inter-rater reliability exceeding 90%. An objective hot flash is defined as a discrete increase in sternal skin conductance of ≧2 umho within a 30-second period followed by a gradual return to baseline. Sternal skin conductance monitoring is more specific to detecting hot flashes than measures of core or peripheral temperature and is highly correlated with self-reported hot flashes under controlled conditions. During the daytime when women are awake, laboratory studies indicate 95% to 98% of subjective hot flashes correspond to objective hot flashes among MW. Unpublished data suggest concordance rates among BCS in a daytime laboratory study were similarly high. Concordance between objective and subjective hot flash frequency is low for nighttime laboratory studies and daytime or nighttime ambulatory studies. Thus, sternal skin conductance monitoring is generally considered to be the gold standard for objective, unbiased hot flash measurement.

Sleep disturbance: The Pittsburgh Sleep Quality Index is a 19-item questionnaire with varying question formats and response categories that produces a global sleep quality and disturbance score based on seven component scores: sleep quality, sleep latency, sleep duration, habitual sleep efficiency, sleep disturbance, use of sleep medications, and daytime dysfunction.(Buys se, et al., Journal of Psychiatric

Research, (1989) 28(2), 193-213. Higher scores indicate poorer sleep quality and more sleep disturbance. Global scores >5 are indicative of poor sleep quality and high sleep disturbance and global scores ≧8 have been linked to daytime fatigue in breast cancer survivors. Psychometrics support reliability and validity among breast cancer survivors. The scale is sensitive to change over time.

Mood disturbance: Mood disturbance is assessed as anxiety and depressive symptoms. Anxiety was assessed with the anxiety subscale of the Hospital Anxiety and Depression Scale_(HADS-A). This is a seven item scale and each item is rated using one of four response options. Higher scores indicate greater anxiety. The scale has been well validated as a measure of anxiety. Depressive symptoms are measured using the Center for Epidemiologic Studies Depression Scale (CESD). This is a 20-item self-report measure assessing presence and severity of depressive symptoms over the past week. Respondents rate each item on a four-point scale. After four positively worded items are reverse scored, responses are summed to obtain total scores ranging from 0 to 60. Scores of 16 and above are indicative of high depressive symptoms. Psychometric properties of the CESD have been extensively examined and the scale has been widely used in research.

Vaginal symptoms: The vaginal symptoms of vaginal dryness and dysparuenia (painful intercourse) are assessed with single items. Women were asked to indicate if they experienced the symptom during the past week, and if so to rate severity from 0 (not at all severe) to 4 (extremely severe). For this analysis we will use a total vaginal symptom severity calculated as the summed severity rating (0 neither symptom/not at all severe to 8 both symptoms extremely severe).

Sample description: Demographic and medical information is being collected from patients and medical records. This includes age, race, ethnicity, menopausal history (e.g., prior hysterectomy, oophorectomy), a full medication history at baseline including prior use of hormone therapy and/or tamoxifen, list of current medications being taken at each time point, breast cancer disease information (stage, date of diagnosis), and breast cancer treatment information (types and dates of all treatments). This information will be used to describe the sample and control for any confounding variables in the analysis.

Sample and settings: Participants are being recruited from clinics associated with the Indiana University Melvin and Bren Simon Cancer Center, the University of Michigan Comprehensive Cancer Center, and the Johns Hopkins/Sidney Kimmel Comprehensive Cancer Center. Women are eligible if they: (a) were post-menopausal, (b) had histologically proven ductal carcinoma in situ (DCIS/stage 0) or stage I-III invasive carcinoma of the breast that was estrogen receptor and/or progesterone receptor positive by immunohistochemical staining, (c) were considering aromatase inhibitor therapy, (d) had completed any adjuvant chemotherapy, (e) were due to receive an aromatase inhibitor as initial adjuvant hormonal treatment or following adjuvant tamoxifen, (f) had an Eastern Cooperative Oncology Group performance status of 0, 1 or 2 (0=fully active, able to carry on all pre-disease performance without restriction, 1=restricted in physically strenuous activity but ambulatory and able to carry out work of a light or sedentary nature, 2=ambulatory and capable of all self-care but unable to carry out any work activities, up and about more than 50% of waking hours), (f) were aware of the nature of their diagnosis and understood the study regimen, its requirements, risks, and discomforts, and (g) were able and willing to provide informed consent. Enrollment of patients will continue through 2009, however, more than 300 patients have been enrolled to date.

A total of 125 women will be included in the proposed study. This will be the first 125 patients enrolled on study, who have completed baseline and one month assessments, and who have complete blood and questionnaire data at both time points.

Data collection schedule and procedures: The data collection schedule pertinent to this proposal is shown below.

| | Baseline prior to | 1 month after |
|---|---|---|
| | starting aromatase | starting aromatase |
| | inhibitor | inhibitor |
| Blood samples collected | X | X |
| Objective hot flash monitoring | 36-hours | 36-hours |
| Sleep disturbance questionnaire | X | X |
| (PSQI) | | |
| Mood disturbance | X | X |
| questionnaires (HADS-A, | | |
| CESD) | | |
| Vaginal symptom severity | X | X |
| rating | | |

Each subject completed a baseline visit (before the start of aromatase inhibitor) and subsequent visits 1, 3, 6, and 12 months later. Each visit involved 36-hour hot flash monitoring, questionnaires (e.g., subjective sleep), and collection of blood samples. Other assessments that were completed at the visits are related to the parent study's primary outcomes and are not relevant to this proposal (e.g., mammography, bone scans, etc.).

Data analysis and interpretation: Plasma orexin concentrations will be entered into excel and transferred to SPSS for analysis. Hot flash and questionnaire data are already double-entered in an ACCESS database and these data will be transferred to SPSS and collated with the plasma orexin data. Using SPSS 15.0, data will be screened to ensure data quality and to evaluate assumptions related to the planned statistical tests (e.g., normality). Descriptive statistics will be computed for all study variables. In addition, Pearson correlations will be computed among all study variables. Psychometric properties of the study measures will be assessed, including test-retest reliability and internal consistency (i.e., Cronbach's alpha). In order to address the specific aims of the present study, we will conduct the following analyses, all of which will be two-sided statistical tests.

Aim1: Compare plasma orexin A levels in breast cancer survivors before and one month after starting aromatase inhibitor therapy.

We will conduct a paired t test comparing the means of orexin A plasma levels at baseline (before starting aromatase inhibitor therapy) and time 2 (one month after starting aromatase inhibitor therapy). A significant t test will suggest a systematic change in orexin A levels after starting aromatase inhibitor therapy. If significant, means and standard deviations at each time point will be evaluated to determine if concentrations increased or decreased.

Aim 2: Examine associations between orexin A plasma levels and menopausal symptoms (hot flashes, sleep disturbance, anxiety, depressive symptoms, vaginal symptoms) in postmenopausal breast cancer survivors before and one month after starting aromatase inhibitor therapy.
a. Orexin A plasma levels will be associated with menopausal symptomatology at baseline prior to starting aromatase inhibitor.

Pearson correlations will be computed among orexin A plasma levels and menopausal symptoms at each time point. Significant correlations among orexin A plasma levels and menopausal symptoms at both time points will suggest a systematic relationship among orexin A and menopausal symptoms.

b. Changes in orexin A will be associated with changes in menopausal symptomatology induced by aromatase inhibitor therapy.

We will conduct a measured-variable path analysis to examine the hypothesized relationships among orexin A plasma levels and menopausal symptoms (hot flashes, sleep disturbance, mood disturbance (anxiety and depressive symptoms, vaginal symptoms) at baseline and time 2. Specifically, a path model will be created with residual changes in orexin A at one month (controlling for orexin A levels at baseline) predicting residual changes in menopausal symptoms at one month (controlling for menopausal symptoms at baseline). Moreover, intercorrelations among menopausal symptoms will be estimated at each timepoint to examine symptom clustering beyond the relationships with orexin A.

The analysis will be conducted using LISREL 8.8, which allows for the testing of theoretical models based on the observed pattern of relationships (e.g., correlations) among a set of measured variables. This type of path analysis offers several advantages over traditional regression analysis, including: the simultaneous testing of all study variables, more accurate estimates of relationships, reduced likelihood of spurious findings, and the ability to handle missing data.

Moreover, the hypothesized model can be evaluated based on how closely it matches the observed pattern of relationships among measured variables. Two goodness-of-fit statistics will be used to evaluate the hypothesized model in this study: the chi-square statistic and the root mean square error of approximation (RMSEA). The chi-square statistic measures the absolute fit between the hypothesized model and the observed pattern of relationships among measured variables. A non-significant chi-square statistic suggests that there is no difference between the hypothesized and observed patterns of relationships and hence, that the hypothesized model is acceptable. The RMSEA statistic adjusts the measure of absolute fit based on the complexity of the hypothesized model, with more complex models receiving a “penalty.” Smaller values of RMSEA indicate better model fit, with values less than 0.06 representing acceptable model fit.

A model with acceptable fit statistics (i.e., nonsignificant chi-square and RMSEA<0.06) will suggest that the hypothesized model is consistent with the observed data. Also, significant beta weights (p<0.05) between residual change in orexin A and residual changes in menopausal symptoms will suggest that changes in orexin A induced by aromatase inhibitor therapy systematically predict changes in menopausal symptoms.

Method for handling missing data: Data will be examined for missing values. If missing values are detected, the pattern of missingness will be evaluated by comparing participants with missing versus complete data on various demographic and study variables. If the data are found to be missing at random, then all data will be included in the path analysis using Full-Information Maximum Likelihood (FIML), which is superior to traditional methods for handling missing data (e.g., listwise or pairwise deletion; Enders, 2001).

### EXAMPLE 4

### Bilateral Ovariectomized Sprague-Dawley Rats as a Model System for Adverse Menopausal activity

Women with surgically induced menopause involving bilateral ovariectomy have a higher frequency of adverse menopausal symptoms such as hot flashes compared to natural transitional follicular degeneration that occurs in natural menopause related to aging. Consistent with this, bilaterally ovariectomizing (OVEX) adult female rats produces measurable adverse menopausal activity such as: 1) circadian disruption of skin (tail) temp (Berendsen et al., (2001) 419(1):47-54); 2) high amplitude tail temp responses to pharmacological challenges (Katovich et al., (1989) Brain Res. 1989;494(1):85-94) and 3) anxiety-like behavior (as measured by open field test and elevated plus maze) (Koss et al., (2004) Horm Behav. 46(2):158-64). These effects can all be attenuated with estrogen replacement. Overall, this supports the use of OVEX as a model of adverse menopausal activity that can be measured objectively.

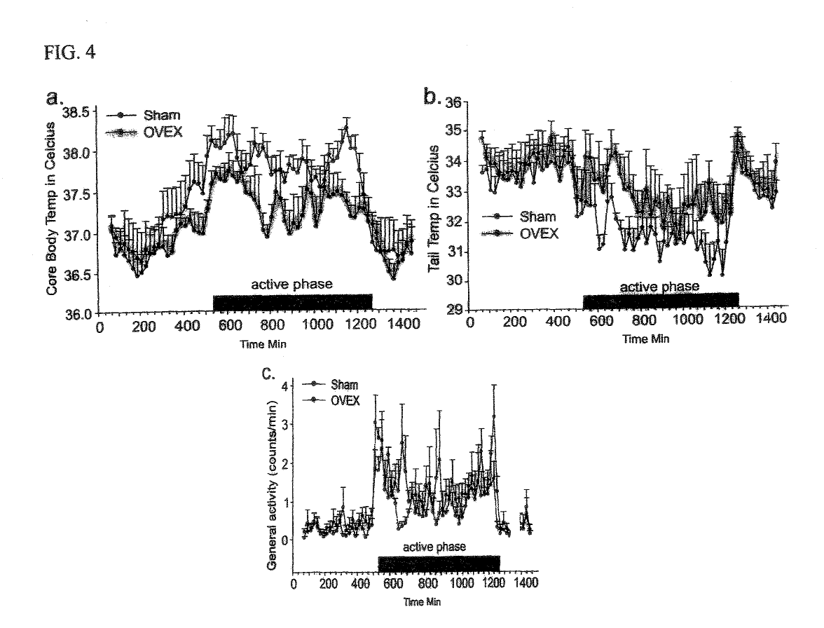

To objectively measure “hot flash” (assessed by tail temp) related activity in female OVEX rats, magnetically activated radiotelemetry probes (dimensions: 3.5 cc volume with 0.05° C. resolution) were implanted subcutaneously under the skin on the back of rat. The probe has 2 leads, each with an ˜1 cm long temp sensor that can be implanted in the tail and into the abdominal cavity to respectively measure skin and core body temp (as well as general ambulatory activity) in freely moving rats for extended periods. Recently we used the dual thermistor probes and were able to replicate previous findings that tail temp decreases during active phase in sham-OVEX rats, but this decrease is blunted in OVEX rats (FIG. 4B).

We observed clear increases in locomotor activity during the active phase (dark period) but no difference between sham OVEX controls and OVEX rats (FIG. 4C). However, we also determined: 1) that core body temp is significantly increased in both sham and OVEX rats during the active phase; 2) but this increase was reduced in OVEX rats (FIG. 4A). This suggests that skin vacomotor activity (which reduces core body temp) is hyperactive in OVEX rats. Furthermore, >1° C. (sometimes >2° C.) spikes in core and tail temp within 60 sec were noted at multiple time points in OVEX, but not sham OVEX rats which may represent spontaneous “hot flash”-associated events. Overall, the use of the dual thermistor probes in OVEX rats provides a model in which to determine the mechanisms and potential non-hormonal treatments for “hot flash”-associated events.

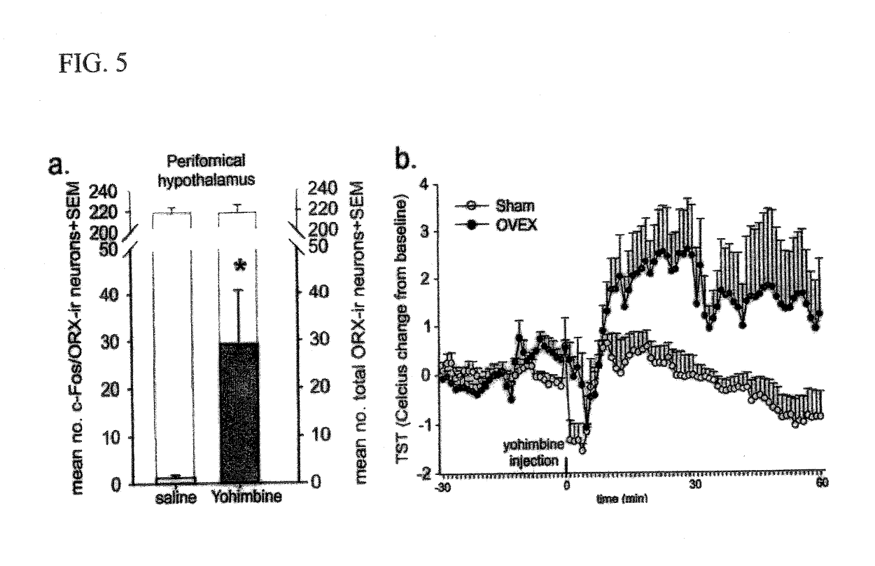

Yohimbine model: There is evidence that suggests that noradrenergic systems are hyperactive following rapid loss of estrogens. Although it is poorly tolerated due to side effects and is not widely used for symptom management, clonidine (alpha2-adrenergic autoreceptor agonist to reduce synaptic release of norepinephrine) can reduce the incidence of “hot flashes” in menopausal women (Freedman et al, Obstet Gynecol, 1990. 76(4): p. 573-8). Conversely, administering intravenous infusions of yohimbine (alpha2-adrenergic autoreceptor antagonist) to symptomatic, but not asymptomatic, menopausal women provokes “hot flashes” (Freedman et al, Obstet Gynecol, 1990. 76(4): p. 573-8). As shown in FIG. 5A, yohimbine injections increase cellular activity in ORX neurons, but also rapidly increase tail skin temp in female OVEX, but not sham OVEX control rats (FIG. 5B). A dramatic drop in core body temp also immediately follows this, which also occurs in women following the onset of a hot flash. This suggests that yohimbine may be provoking menopausal symptoms through the ORX system.

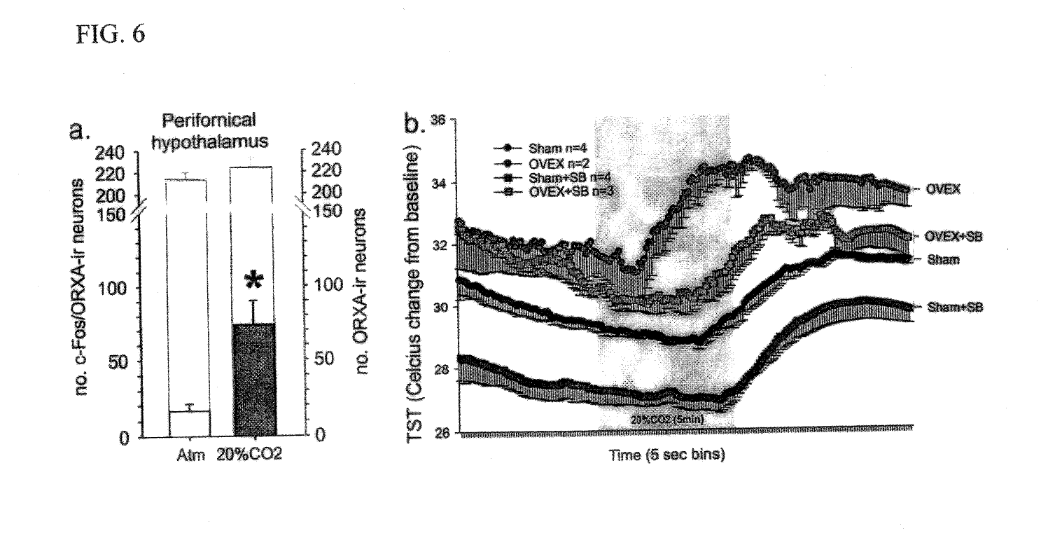

Hypercapnia model: There is also evidence that altered respiration patterns may trigger hot flashes whereas paced respiration may alleviate hot flash incidence and intensity. Increased or decreased respiration activity alters blood CO₂/pH levels, and a hypersensitivity to CO₂/pH may be present in conditions where estrogen activity is very low. For instance, blood pH is reduced during a “hot flash” (Aktan et al., Maturitas, 1998. 29(3): p. 225-7) and menopausal women undergoing paced respiration have significant reductions in hot flash frequency (Freedman and Woodward, Am J Obstet Gynecol, 1992. 167(2): p. 436-9). Overall, this suggests respiration changes that induce hypercapnia may trigger hot flashes. Consistent with this, briefly exposing rats to a 5 min challenge of normoxic hypercapnic (20% CO₂) gas, which increase activity in ORX neurons (FIG. 6A), causes a flushing response which occurs faster and at a higher intensity in OVEX rats, compared to sham-OVEX controls (FIG. 6B). A dramatic drop in core body temp also immediately follows this, which also occurs in women following the onset of a hot flash. More importantly, pretreating OVEX rats with an ORX 1 receptor antagonist delays the onset of the hot flash and reduces the intensity. The ORX 1 receptor antagonist reduced baseline levels of tail temp in controls which suggests that the antagonist is inducing cutaneous vasoconstriction that is associated with estrogen replacement (Fraenkel et al., Ann Intern Med, 1998. 129(3): p. 208-11). This also suggests that women diagnosed with chronic obstructive pulmonary disorder or suffering from conditions with bronchoconstriction (e.g., asthma) may have increased vulnerability to menopausal symptoms such as hot flashes that would be exacerbated with conditions or pharmacological treatments that reduce estrogen activity.

Based on these results it is believed that OVEX or pharmacological estrogen blocking treatments produce measurable menopause-associated anxiety behavior and diurnal disruption of tail and core temp and exacerbated tail temp increases following provocation. These effects are further exacerbated by the administration of tamoxifen. Accordingly, OVEX or pharmacological estrogen blocked rats can serve as a valid rat model of multiple adverse menopausal-related symptoms. Furthermore, it is believed that that the orexin 1 receptor antagonists attenuate the incidence and severity of menopausal symptoms resulting from an OVEX and/or tamoxifen and represent a novel, fast acting, non-hormonal treatment for menopausal symptoms. Accordingly it is anticipated that orexin 2 and dual orexin 1 and 2 receptor antagonists would also attenuate the incidence and severity of menopause associated symptoms since the orexin 2 receptor is colocalized with the orexin 1 receptor site at most brain regions associated with regulating anxiety and temperature regulation; and activating the orexin 2 receptor at those brain regions evokes similar responses as activating the orexin 1 receptor, albeit with a less potent effect.

To further investigate ORX receptors' role in menopausal-associated activity, menopausal states in adult female rats will be induced by either removing the ovaries (ovariectomy: OVEX) and/or inhibiting estrogen activity with tamoxifen to model a menopausal state. Adverse menopausal activity will be assessed 12 days post OVEX (or control sham-OVEX) surgeries +/−daily subcutaneous injections of 10 mg/kg tamoxifen (or control vehicle) from the following: 24 hr tail skin and core body temp and sleep-related locomotor activity will be assessed by using radio-telemetry probes; followed by anxiety behavior testing on day 13; and “hot flash” provocation (assessed by tail and core body temp) following yohimbine or hypercapnia challenge. We will then determine if menopausal-associated activity can be attenuated by systemic injections of a centrally active ORX 1 receptor antagonist.

### EXAMPLE 5

A variety of studies indicate that ovarian hormones influence anxiety in female rodents primarily through the ERβ, whereas within the brain the ERα primarily regulates reproductive behaviors. Treating OVEX'ed rats (menopause-like condition) with DPN (diarylpropionitrile, an ERβ selective agonist) or E2 decreased anxiety behaviors, whereas PPT (Tris(4-hydroxyphenyl)-4-propyl-1Hpyrazole, an ERα selective agonist) increase anxiety behaviors in the elevated plus maze (EPM) and open field tests (OFT). Consistent with this, ERβ knockout females display higher levels of anxiety than wild type littermates in the EPM (Imwalle et al., (2005) Physiol Behav 84: 157-163). Taken together, these findings indicate that ERβ is important in mediating the anxiolytic effects of estrogens on anxiety. ERβs, but not ERα's, are also expressed in the ORX hypothalamic region and may explain the neural circuits thru which estrogen's exert their anxiolytic effects.

This study will be conducted to determine the effect of 17-β-estradiol (E2) replacement, or selective Eα or Eβ receptor (ERα, ERβ) agonists on: 1) ovariectomy (OVEX)-induced menopausal symptoms; and 2) ex vivo ORX activity (i.e., expression of ORX and ORX 1 and 2 receptor protein and mRNA in the brain, and ORXA concentrations in plasma).

Research Design—Baseline weight and anxiety behavior (i.e., OFT and EPM) will be assessed on adult female Sprague-Dawley rats prior to surgeries involving: 1) bilateral OVEX or sham surgical OVEX; and 2) abdominal implantation of a radiotelemetry probe (Data Sciences) to assess 24 hr locomotor activity (sleep disruption) and core body temperature [CBT: a hot flash predictor (Tataryn et al., 1980; Tataryn et al., 1981)]. Rats will receive daily subcutaneous neck injections of: 0.2 ml vehicle control; ET [0.25 mg/kg of 17-β estradiol (E2), Sigma]; ERβ agonist [1.0 mg/kg DPN, Tocris]; or an ERα agonist [1.0 mg/kg PPT, Tocris]. Recent data show that these doses of E2 and ERβ agonist (DPN), but not the ERα agonist (PPT) block the anxiety induced by OVEXing a female rat (Walf et al., 2010). On day 9 post OVEX, 24 hr monitoring of activity and CBT will occur 1 hr before onset of inactive sleep cycle and stop 1 hr post active phase. Anxiety testing will occur on day 11 post OVEX where our group has previously shown increases in anxiety. On D12 ORX-A protein concentrations will be assess in the hypothalamus (central activity) and in plasma (peripheral activity), since ORXA crosses the BBB with ease using an RIA (Phoenix Pharmaceuticals), which has been successfully used previously. ORX precursor and ORX 1 and 2 receptor mRNA will be assessed using RT-PCR in micropunched brain regions implicated in anxiety, sleep disruption and temperature/vasomotor regulation. Plasma E2 concentrations will be verified using a RIA (Diagnostics Systems Labs).

### Prediction and Relevance

Estrogen depletion is anticipated to increase ORX and ORX receptor expression within the brain and ORXA in the plasma, that will be correlated with an increase in adverse menopausal symptoms post OVEX. We anticipate that the E2 and ERβ, but not ERα, receptor agonist will attenuate OVEX-induced increases in menopausal symptoms and central ORX activity. Overall, this will determine: 1) the link between OVEX-induced menopausal symptoms and ORX activity; 2) if selective ERβ agonists can attenuate menopausal symptoms by restoring ORX activity to control levels, and 3) if plasma ORX could be used to assess central activity.

### EXAMPLE 6

### Determine the Effect of Silencing the ORX Precursor Gene in the Hypothalamus on Menopausal Symptoms Following an OVEX in Female Rats

It is anticipated that silencing the ORX precursor in the hypothalamus following an OVEX (a model of peri-menopause) will: 1) attenuate OVEX-induced adverse menopausal baseline anxiety and circadian disruption of skin (i.e., tail temp) and core body temp and locomotor activity; and 2) prevent acute “hot flash” (measured by skin temp) provoked by raising the temp of ambient environment.

### Research Design

Baseline weight and anxiety behavior (i.e., open field test: OFT) will be assessed on adult female Sprague-Dawley rats prior to surgeries involving: 1) bilateral OVEX or sham surgical OVEX; and 2) abdominal implantation of a radiotelemetry probe (Data Sciences) to assess 24 hr locomotor activity (sleep disruption) and tail skin and core body temperature (hot flash predictors). On day 7 bilateral cannula will be stereotaxically implanted into the dorsomedial/lateral hypothalamus (DMH/LH). On day 10 small interfering (si) siRNA for preproORX gene [100 nMol/500 nl injection, Dharmacon] or control siRNA will be injected into the DMH/LH (which we previously have shown reduces local orexin protein by ˜80% without inducing sleep associated behavior (Johnson et al., (2010) Nature medicine 16(1):111-5.12). On day 12, 24 hr monitoring of tail and core body temp and locomotor activity will occur 1 hr before onset of inactive sleep cycle and stop 1 hr post active phase. Anxiety testing (OFT+elevated plus maze: EPM) will occur on day 13 post OVEX following by raising ambient temp in the homecage to provoke “hot flash”-related changes in tail (skin) temp which provokes “hot flashes” in symptomatic postmenopausal women Gene silencing in the DMH/LH will be confirmed ex vivo by measuring orexin protein (radio-immunoassay) and mRNA (RT-PCR). Phase of estrous of ShamOVEX rats will be assessed by daily vaginal smears and take blood samples (via trunk blood) at end of the experiments for ex vivo analyses of estrogen levels using a radio-immunoassay (Diagnostics Systems Laboratories Inc.).

Silencing the orexin gene in the hypothalamus will reduce local orexin expression and is anticipated to attenuate OVEX-induced increases in menopausal symptoms. Overall, this will determine if orexin plays a role in OVEX-induced menopausal symptoms and a potential nonhormonal target for the treatment of adverse menopausal symptoms.

### EXAMPLE 7

### Determining the Effect of Selective ORX1 or ORX2 Receptor Antagonists on Menopausal Symptoms Following an OVEX in Female Rats

Menopausal states in adult reproductive female rats will be induced by removing ovaries (ovariectomy: OVEX) to model a menopausal state. We will then determine if menopausal-associated activity can be attenuated by: 1) silencing the ORX precursor gene in the hypothalamus; 2) systemic injections of a centrally active ORX 1 or 2 receptor antagonists. Adverse menopausal activity will be assessed by 2 experimental designs 12 days post OVEX or control sham-OVEX surgeries as follows:

1) 24 hr “hot flash” and sleep disturbance-related activity will be assessed by using radio-telemetry thermister/activity probes (data sciences international) to respectively assess thermoregulatory peripheral tail temp and core body temp and locomotor activity. On following day anxiety behavior testing (open field test then elevated plus maze) will occur followed by;

2) provoking “hot flash” (assessed by radiotelemetry thermistors to assess core and tail temp) by raising ambient temp. Ex vivo analyses of hypothalamic ORX protein and mRNA and blood levels of estrogen will occur where appropriate.

Treating OVEX rats (a model of peri-menopause) with ORX 1 and 2 receptor antagonists will: 1) attenuate OVEX-induced adverse menopausal anxiety and circadian disruption of cutaneous (i.e., tail temp) and core body temp and locomotor activity; and 2) prevent acute “hot flash” (measured by skin temp) provoked by raising ambient temperature.

### Research Design

OVEX rats will receive an intraperitoneal injection of either an orexinl receptor inhibitor (e.g., 30 mg/kg, SB334867), a dose we showed to block anxiety, hyperthermic, and locomotor responses in an animal model of panic, see FIGS. 1 and 2 that crosses blood brain barrier and remains centrally active >4 hrs; or an orexin 2 receptor (e.g., 30 mg/kg, TCSOX229, Tocris) antagonist prior to the onset of the active and inactive phase of the circadian cycle on day 12. On D13 rats will receive the orexin 1 or 2 receptor antagonist injection 30 min prior to anxiety assessment and “hot flash” provocation.

It is anticipated that the orexin 1 and 2 receptor antagonists will attenuate the severity of menopausal symptoms resulting from an OVEX (anxiety-like behavior; circadian disruption of skin and core body temp and locomotor activity; and hot flash vulnerability) and provide a novel, fast acting, non-hormonal treatment for menopausal symptoms. This is especially relevant since there are 2 dual orexin receptor antagonists [Almorexant (Neubauer D N (2010) Curr Opin Investig Drugs. 11(1):101-10) and MK-4305 (Cox, et al., (2010) J Med Chem 53(14):5320-32] in phase III trials that are safe, have few side effects, and are currently under investigation for insomnia.

Data Analysis: Dependent variables for analyses (i.e., OFT, EPM, wt, CBT, and activity, ORX E2 measures) will be analyzed using a two way ANOVA with OVEX+ER, SERM, or ORX receptor antagonists as the main factors and time as a repeated measure. In the presence of significance, post-hoc tests will be conducted using a Tukey's and Dunnet's test for within group and time effects (respectively) or unpaired or paired t-tests when appropriate.

### EXAMPLE 8

### An Rodent Model of Adverse Menopausal Related Symptoms

Women with surgically induced menopause involving bilateral ovariectomy have a higher frequency of adverse menopausal symptoms such as hot flashes compared to natural transitional follicular degeneration that occurs in natural menopause related to aging. Consistent with this, bilaterally ovariectomizing (OVEX) adult female rats produces measurable adverse menopausal activity such as: 1) circadian disruption of skin (tail) temperature (Berendsen et al., 2001, Eur J Pharmacol 419:47-54); 2) high amplitude tail temp responses to pharmacological challenges (Katovich et al., 1989, Horm Behav 46:158-164) and 3) anxiety-like behavior (as measured by open field test and elevated plus maze) (Koss et al., 2004, Horm Behav 46:158-164). These can all be attenuated with estrogen replacement and also with an orexin 1 receptor antagonist (SB334867)(see FIG. 1b). Overall, this supports the use of OVEX as a model of adverse menopausal activity that can be measured objectively measured.

To assess “hot flash” related activity we implant magnetically activated state of the art radiotelemetry probes (F40-TT, Data Sciences International, Inc.; dimensions: 3.5 cc volume with 0.05° C. resolution) into the abdomen of the rat. The probe has 2 leads, each with an ˜1 cm long temp sensor that can be implanted in the tail and into the abdominal cavity to respectively measure skin and core body temp (as well as general ambulatory activity) in freely moving rats for extended periods. This gives us the ability to assess: 1) acute “hot flash” related increases in tail temperature following a triggers that provoke “hot flashes” in women with menopause (e.g., pharmacological; hypercapnic gas; or increases in ambient temp); or 2) long term aberrant skin vasomotor activity (tail temp) and sleep disruption in our animal model of menopausal symptoms for up to a month. Rapid assessment of “hot flash” related increases in tail temp are also assessed using a thermistor lead taped (moleskin tape) to the ventral surface of the tail approximately 1 cm from base of tail.

1. A method of treating a female patient for menopausal symptoms, said method comprising the step of
administering to said patient a composition comprising an inhibitor of orexin activity, in an amount sufficient to prevent the onset, or reduce the severity, frequency or duration of said menopausal symptoms.

2. The method of claims 1 further comprising the step of identifying women with below average estrogen levels, wherein such women are administered said composition prophylactically or in response to symptoms.

3. The method of claim 1 wherein the composition comprises an ORX1 receptor antagonist.

4. The method of claim 1 wherein the composition comprises an ORX2 receptor antagonist.

5. The method of claim 1 wherein the composition comprises ORX1 and ORX2 receptor antagonist activity.

6. The method of claim 1 wherein the composition comprises an orexin inhibitor selected from the group consisting of SB334867, MK4305 and Almorexant.

7. The method of claim 1 wherein the patient is receiving aromatase inhibitor therapy.

8. The method of claim 1 wherein the patient is menopausal.

9. A method of treating a woman to prevent or reduce the severity, frequency or duration of hot flashes, said method comprising the steps of
identifying women that have below average estrogen levels;
administering to woman having below average estrogen levels a composition comprising an orexin inhibitor.

10. The method of claim 9 wherein the orexin inhibitor interferes with ORX receptor activity.

11. The method of claim 9 wherein the composition comprises an orexin inhibitor selected from the group consisting of SB334867, MK4305 and Almorexant.

12. The method of claim 1 wherein said patient is breast cancer or ovarian cancer patient, said method comprising
identifying said cancer patients receiving aromatase inhibitor therapy or other drugs that block the effects of estrogen at the receptor,
administering to said identified cancer patient a composition comprising an inhibitor of orexin activity to prevent the onset, or reduce the severity, frequency or duration of menopausal symptoms.

13. (canceled)

14. The method of claim 12 wherein the inhibitor is an orexin receptor antagonist selected from the group consisting of SB334867, MK4305 and Almorexant.

15. The method of claim 12 wherein the orexin receptor antagonist is administered prior to the administration of the aromatase inhibitor therapy.

16. The method of claim 12 wherein the composition comprises an inhibitor of orexin A activity.

17. The method of claim 16 wherein the inhibitor is SB334867.

18-20. (canceled)

21. A method for identifying candidate compounds for treating menopausal symptoms, said method comprising
providing a freely moving female rodent menopausal model;
administering a test compound to said rodent menopausal model;
monitoring the tail temperature of said rodent menopausal model during the animal's transition from a behavioral inactive to active phase; and
identifying test compounds as a candidate compounds when the test compound upon administration reduces rodent menopausal model tail skin temperature relative to a rodent menopausal model not receiving said test compound.

22. The method of claims 21 wherein the rodent menopausal model is an bilaterally ovariectomized (OVEX) adult female rodent.

23-30. (canceled)
